(19) United States
(12) Patent Application Publication
(10) Pub. No.: US 2009/0054622 A1
(43) Pub. Date: Feb. 26, 2009

(54) Peptide variants of the tumor marker MUC1 and their application

(76) Inventors: Franz-Georg Hanisch, Koln (DE); Stefan Muller, Bergisch-Gladbach (DE)

Correspondence Address: BUCHANAN INGERSOLL & ROONEY PC, P.O. BOX 1404, ALEXANDRIA, VA, 22313-1404, US

(21) Appl. No.: 09/931,638

(22) Filed: Aug. 16, 2001

Related U.S. Application Data
(63) Continuation of application No. PCT/DE00/00440, filed on Feb. 17, 2000.

(30) Foreign Application Priority Data
Feb. 17, 1999 (DE) 199 06 615.9

(51) Int. Cl. C07K 14/00

(52) U.S. Cl. 530/326

(57) ABSTRACT

The invention refers to peptide variants of the tumor marker MUC1 and their application in antigenic and immunogenic remedies. It concretely refers to peptide variants of the MUC1 tandem repeat unit within the VNTR (=variable number of tandem repeats) domain.

The invention refers to peptide variants of the tumor marker MUC1 and their application in antigenic and immunogenic remedies. It concretely refers to peptide variants of the MUC1 tandem repeat unit within the VNTR (=variable number of tandem repeats) domain.

Epithelial mucins are glycoproteins with repetitive amino acid sequences and a high carbohydrate content, which can be membrane-bound or secreted and occur in many glandular epithelia. The best characterized epithelial mucin is the membrane-bound MUC1, also described as PEM, PUM, EMA, MAM-6, PAS-0 or episialin (Finn, O. et al., Immunol. Rev. 145: 61, 1994).

MUC1 per se is not a tumor-specific molecule; its suitability as a tumor antigen is based on qualitative and quantitative alterations of the carbohydrate content in tumors (Burchell, J., and Taylor-Papadimitriou, J, Epith. Cell Biol. 2: 155, 1993). Thereby new epitopes are exposed, which are recognized by the immune system (humoral and cellular defense).

The extracellular portion of MUC1 consists of a variable number of repeating 20 amino acid (aa) units, the so-called “tandem repeats”. This MUC1 tandem repeat unit of the VNTR domain was described on the DNA level by Gendler et al., J. Biol. Chem. 265: 15286-15293, 1990, to correspond to an icosapeptide with the sequence PAPGSTAPPAHGVTSAPDTR (PAP20) and was registered under accession numbers J03651, J05288, and J05581 at the GenBank™/EMBL (refer also to the literature given under these entries). This sequence is regarded to be highly conserved in humans, since up to now no structural variants were found.

The invention was based on the search for peptide variants, which could serve for the generation of specific immunodiagnostic reagents.

Surprisingly, novel features of the MUC1 peptide structure were revealed by sequencing analyses on the protein and DNA levels, which were obtained for secretary MUC1 from normal breast epithelium (milk) and a variety of carcinoma cells.

The invention is based on the finding that MUC1 isolated from milk does not exhibit variation of the established PAP20 sequence, while human carcinoma cells display alternative sequences of the VNTR peptide at high incidence.

According to the invention three amino acid replacements were detected in the PAP20 sequence:

### Pro 9→Ala, Asp18→Glu, Thr19→Ser,

which were identified by mass spectrometry and quantitative Edman degradation (see performance example).

Objects of the invention are, accordingly, peptide variants of the 20 aa MUC1 repeat peptide within the VNTR domain (variable number of tandem repeats), which deviate from the known VNTR peptide sequence at positions 9 and/or 18 and/or 19.

Especially peptides with the SEQ ID No.1:

### PAPGSTAPAAHGVTSAPDTR (PAP20-A)

with the SEQ ID No.2:

### PAPGSTAPPAHGVTSAPESR (PAP20-ES)

and with the SEQ ID No. 3:

### PAPGSTAPAAHGVTSAPESR (PAP20-AES)

are referred to.

The Pro→Ala substitution has a strong influence on the secondary structure of the peptide, since proline residues contribute mainly to the formation of a left-handed poly-L-proline type-II helix. The structural alteration introduced by the Pro X Ala substitution influences the antigenicity of the VNTR peptide. This holds particularly also true for the PDTR motif within the repeat unit, which represents an internally stabilised structural element with the characteristics of a bump (knob) and serves as an immunodominant target. The (conservative) replacement of two aa within this motif (Asp18-Thr19→Glu18-Ser19) should influence the conformation via altered lengths of the adjacent aa side chains and hence should modify its antigenicity and immunogenicity. Since specific aa replacements within the VNTR domain were only detected in tumor MUC1, the altered peptide epitopes have a high immunodiagnostic and tumortherapeutic potential.

The peptides can be prepared according to established methods by solid-phase synthesis or by genetic engineering.

The invention-defined peptides can be used in the development of immunoreagents, in terms of specific immunodiagnostic substances or tumor vaccines. These contain at least one of the invention-defined peptides.

The most effective adjuvant-based immunotherapy is vaccination. Two prerequisites are required:

1) a suitable target epitope should be present on tumor cells;
2) it should be possible to generate a highly immunogenic, preferentially synthetic form of a vaccine.

According to the invention, tumor vaccines are generated on the basis of the molecular structure of human epithelial mucin, MUC1, which serve preferentially for the combat of “minimal residual disease” after surgical treatment or an other primary therapy and which contain the MUC1-derived peptide variants of the VNTR domain, preferentially peptides SEQ ID No. 1, 2, and/or 3.

Such a tumor vaccine can be used in the defense against tumor cells preferably from breast, colorectal or pancreatic carcinomas, in terms of an “active specific immunisation”.

Moreover, the invention concerns analytical methods for the determination of identity and incidence of DNA mutations, which underlie the above specified peptide variants.

The development of test kits on the basis of these DNA mutations could enable the definition of prognostic parameters for tumor diagnosis. The test principle is based on a PCR-ELISA (polymerase-chain-reaction-enzyme-linked immunoassay) using amplified VNTR domains from genomic DNA.

The detection of variant peptides should, according to the invention, serve for the optimization of therapeutic approaches.

### EXAMPLE

Using the human breast cancer cell line T47D as an example it will be explained, which evidence could be obtained for the occurrence of alternative VNTR peptide sequences.

The secretory glycoform of MUC1 was isolated from culture supernatants of the cell line by affinity chromatography on immobilized anti MUC1 antibody (BC3). After partial enzymatic deglycosylation (α-sialidase, β-galactosidase) the VNTR domain of the mucin was fragmented into icosapeptides (PAP20) by cleavage with the Arg-C specific endoproteinase clostripain. The rpHPLC purified glycopeptides were sequenced by mass spectrometry (QTOF-ESI-MS) and quantitative Edman degradation. The combined data can be unequivocally interpreted by assuming that

(1) all five positions within VNTR peptide are glycosylated
(2) besides the known PAP20 sequence three alternative icosapeptides with an incidence of >50% of the total repeat peptides are present.

While the non-conservative replacement Pro9→Ala occurs also independently and is found in about 30% of the VNTR peptides, the Asp18→Glu and Thr19→Ser replacements occur concertedly in about 50% of the VNTR peptides. A comparable high incidence of these replacements was detected on the DNA level for a variety of other human carcinoma cells. Besides the known polymorphism of MUC1 referring to the number of tandem repeats a further genetic polymorphism is indicated to exist on the level of the peptide sequences.

1) Peptide variants of MUC1 within the VNTR (variable number of tandem repeats) domain comprising 20 aa which deviate at positions 9 and 18 and 1 or 19 from the well-known VNTR domain.

2) Peptide variants according to claim 1, characterized by the SEQ ID No, 2 sequence PAPGSTAPPAHGVTSAPESR (PAP20-ES).

3) Peptide variants according to claim 1, characterized by the SEQ ID No. 3 sequence PAPGSTAPAAHGVTSAPESR (PAP20-AES).

4) Immunogenic substances/reagents characterized by their content of at least one of the peptides specified under claims 1 [symbol] or the SEQ ID No. 1 sequence PAPGSTAPAAHGVTSAPDTR (PAP20-A).

5) Antigenic substances/reagents characterized by their content of at least one of the peptides specified under claims 1 [symbol] or the SEQ ID No 1 sequence PAPGSTAPAAHGVTSAPDTR (PAP20-A).

6) Test kit for the determination of identity and incidence of DNA mutations within the VNTR domain, which contains at least one of the primer sequences corresponding to one of the peptides specified under claims 1 to [symbol] or the SEQ ID No. 1 sequence PAPGSTAPAAHGVTSAPDTR (PAP20-A).

7) Use of these peptides specified under claims 1 [symbol] or the SEQ ID No, 1 sequence PAPGSTAPAAHGVTSAPDTR (PAP20-A) for the generation of cancerostatic remedies, especially for the generation of effective vaccines in a tumor therapeutic context.


SEQUENCE LISTING (3 sequences)

SEQ ID NO: 1 - protein, 20 aa, Artificial Sequence
  designed peptide, variant of MUC1 - PAP20-A
PAPGSTAPAAHGVTSAPDTR

SEQ ID NO: 2 - protein, 20 aa, Artificial Sequence
  designed peptide, variant of MUC1 - PAP20-ES
PAPGSTAPPAHGVTSAPESR

SEQ ID NO: 3 - protein, 20 aa, Artificial Sequence
  designed peptide, variant of MUC1 - PAP20-AES
PAPGSTAPAAHGVTSAPESR